(19) United States
(12) Patent Application Publication
(10) Pub. No.: US 2020/0268929 A1
(43) Pub. Date: Aug. 27, 2020

(54) SETTABLE SILICON NITRIDE CEMENTS

(71) Applicant: CTL Medical Corporation, Addison, TX (US)

(72) Inventors: Sean SUH, Milltown, NJ (US); Jon SUH, Ambler, PA (US); Danny CHON, Addison, TX (US)

(21) Appl. No.: 16/799,248

(22) Filed: Feb. 24, 2020

Related U.S. Application Data
(60) Provisional application No. 62/809,410, filed on Feb. 22, 2019.

(51) Int. Cl. A61L 24/00; C08K 3/34; C08L 33/12; A61L 24/06; A61L 24/02; C08K 7/18

(52) U.S. Cl. CPC A61L 24/0089; A61L 2300/404; C08L 33/12; A61L 24/06; A61L 24/02; A61L 24/0015; C08K 7/18; C08K 2201/003; C08L 2205/02; C08L 2203/02; A61L 2300/112; A61L 2300/10; A61L 2300/414; A61L 2300/412; C08K 3/34

(57) ABSTRACT

Disclosed are settable bone cements incorporating silicon nitride in various forms as a component, including powders, granules, particulates, portions, layers and/or coatings of solids and/or particulates of silicon nitride and/or components thereof, that may be useful in joint and/or bone replacement implants used in spinal surgeries, dental surgeries and/or other orthopedic and/or general surgical procedures.

### CROSS-REFERENCE TO RELATED APPLICATIONS

This application claims priority to and benefit of U.S. Provisional Patent Application No. 62/809,410 entitled “SI3N4 MIXED BONE CEMENT AND RESORBABLE GRANULE” filed Feb. 22, 2019, the disclosure of which is incorporated by reference herein in its entirety.

### FIELD OF THE INVENTION

The present subject matter relates generally to settable bone cements incorporating silicon nitride in various forms as a component, including powders, granules, particulates, portions, layers and/or coatings of solids and/or particulates of silicon nitride and/or components thereof, that may be useful in joint and/or bone replacement implants used in spinal surgeries, dental surgeries and/or other orthopedic procedures. In various embodiments, the settable bone cement may assume an initial flowable and/or moldable stage followed by a thickened, more solidified and/or cured stage, allowing the material to be injected and/or shaped into more rigid shapes and/or forms that can withstand compression, tension, lateral strain and/or various combinations thereof without failing.

### BACKGROUND OF THE INVENTION

Bone cement compositions are useful in applications such as dental and medical procedures. In particular, bone cements are frequently used to fill voids in natural bony structures as well as in bonding or affixing implants and implant materials to natural bone and/or to repair damaged natural bone. Typically, current bone cement compositions are sold in two-part preparations containing a powder (or dry) part and a liquid (or wet) part, which, when combined, polymerize to form a hardened substance mimicking many of the physical properties of natural bone. The powder part typically includes a polymeric material, such as acrylate polymers (i.e., a powered MMA-styrene co-polymer), while the liquid part includes a reactive monomer, such as methyl methacrylate.

While PMMA bone cements have been quite successful in medical use, these cements can also have numerous drawbacks. For example, the polymerization of bone cement is an exothermic process that releases significant heat energy, which can cause tissue necrosis. The liquid methyl methacrylate in PMMA is toxic and can induce hypotensive effects, which in some cases can lead to cardiac arrhythmias or ischemic myocardium. If introduced and/or injected in a “runny” or too thin consistency, liquid PMMA cement may leak into surrounding soft tissues, into veins and/or arteries of the bloodstream, and along nerve channels—where the PMMA can then harden and potentially create embolisms and/or cause tissue and/or nerve damage. Moreover, PMMA bone cements typically cannot be degraded, they lack biological activity, and cannot form osseous bonding with host bone tissue. After being implanted in vivo, PMMA bone cements are poor in integrating with the surrounding bone tissue and not conducive to bone cell adhesion and growth.

Another common complication of cemented arthroplasty is cement fragmentation and foreign body reaction to wear debris, resulting in prosthetic loosening of the cemented prosthesis and/or periprosthetic osteolysis. In many cases, mechanical weakness in the bone cement, primarily attributed to the addition of barium sulphate and zirconium oxides (for radiological detection) and/or the addition of antibiotics to the bone cement, can significantly increases the risk of cement cracking, bond failures, debris generation and/or implant loosening.

In spite of its numerous clinical disadvantages, PMMA remains highly popular with surgeons and other physicians. While there have been various attempts to improve PMMA performance by the incorporation of various additives, such attempts have met with limited success in addressing many of PMMA's drawbacks. It would be desirable, therefore, for a fast setting bone cement to be developed with a performance and/or failure profile similar to natural bone structures, with improved osteo-conduction, osteo-induction and/or the potential for osteo-integration with surrounding tissue structures.

### BRIEF SUMMARY OF THE INVENTION

In accordance with various aspects of the present subject matter, bone cement formulations are described that incorporate silicon nitride (i.e., Si₃N₄ and/or chemical analogues thereof) in their mixtures and/or composition, which may include the incorporation of silicon nitride powders, granules, particulates, portions, pebbles, blocks, layers and/or coatings of solids and/or particulates within the bone cement mixture. In various embodiments, the bone cement may be a liquid, paste, gel or dough, and preferably hardens to a substantially solid solidified material.

In at least one exemplary embodiment, a PMMA bone cement formulation comprising a powered MMA-styrene co-polymer and a reactive monomer such as methyl methacrylate can be combined with various percentages by weight and/or volume of a ceramic material such as a silicon nitride material, which when mixed and polymerized can result in a polymerized and/or “cured” block, implant and/or structure capable of implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

In at least one embodiment, a PMMA bone cement formulation comprising a powered MMA-styrene co-polymer and a reactive monomer such as methyl methacrylate can be combined with various percentages by weight and/or volume of a powdered, granulated and/or fluidized silicon nitride material, which when mixed can create a flowable and/or moldable material which will desirably harden and/or polymerize into a variety of shapes, which can include injection of the flowable material through a syringe or tube into a void or opening to partially and/or fully fill the void or opening, wherein the material will subsequently harden and/or polymerize into a shape which can be defined by the cavity in which it sits. This could include the injection into various anatomical locations as well as injection and/or introduction into implants and/or other devices prior to, during and/or after their implantation into a targeted patient anatomy (i.e., such as within the graft chamber of an intervertebral fusion implant).

In accordance with various aspects of the present subject matter, bone cements and/or other implants, devices and/or components thereof are described that incorporate silicon nitride (i.e., Si3N4 and/or chemical analogues thereof) in their construction, either in the entirety of the implant as well as components, portions, layers and/or surfaces thereof. In various embodiments, the silicon nitride material(s) will be highly osteo-inductive and/or osteoconductive and will desirably facilitate and/or promote fixation to adjacent living bone surfaces, while concurrently reducing and/or inhibiting periprosthetic infection and/or bacterial adhesion.

In various applications, the utility of silicon nitride as an implant material can be enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

If desired, implants can be constructed from a variety of modular components, including modular components comprising different materials and/or injectable or formable silicon nitride/PMMA cements. If desired, such modular components could be provided in a kit form for selection and/or assembly in a surgical theatre and/or in situ during a surgical procedure. If desired, various components may be removable and replaceable.

Various surgical methods for preparing anatomical surfaces and/or for implanting or placement of the various devices and/or components described herein are also described, including the insertion and placement of implants between adjacent vertebrae of the spine as well as within bones and/or between bones and/or joint surfaces or other body locations.

In accordance with another aspect of the present subject matter, various methods for manufacturing devices and/or components thereof, as set for within any of the details described with the present application, are provided.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 illustrates a cross-sectional view of a vertebral bone filled with a PMMA cement;

FIG. 2 illustrates a cross-sectional view of a vertebral bone filled with one exemplary embodiment of a silicon nitride cement;

FIGS. 3A and 3B depict perspective views of cement structures incorporating resorbable silicon nitride granules;

FIGS. 4A and 4B depict the cement structures of FIGS. 3A and 3B after absorption; of some silicon nitride;

FIG. 5A depicts a perspective view of a silicon nitride agglomeration in a cement formulation;

FIG. 5B depicts various exemplary geometries for resorbable silicon nitride granules mixed into a PMMA cement formulation to enhance macro porosity;

FIGS. 6A through 6C depict various exemplary silicon nitride granular shapes;

FIG. 7 depicts an exemplary grain size distribution for silicon nitride granules for use in cement formulations;

FIGS. 8A and 8B depict SEM photographs of an exemplary PMMA cement incorporating resorbable ceramic granules;

FIG. 8C depicts an exemplary ceramic granule with associated PMMA cement;

FIG. 9 depicts various cross-sectional views of a spinal implant with various exemplary silicon nitride cemented insert geometries formed therein;

FIG. 10 depicts exemplary degrees of hydrophobicity for various medical grade materials, including a silicon nitride cement;

FIGS. 11A and 11 B depict cross-sectional views of silicon nitride cement surfaces with neovascularization induced within the porous sections of the implant;

FIGS. 12A through 12C depict three exemplary implants made of PEEK, Titanium and a silicon nitride cement and their effects on adjacent living bone;

FIG. 12D depicts a magnetic field induced by a bar-type magnet;

FIG. 12E depicts the effect of silicon nitride material on new bone growth;

FIGS. 13A through 13C depict exemplary effects of a silicon nitride surface on bacteria near the implant; and

FIGS. 14A through 14F depict various desirable attributes of an implant comprising a silicon nitride cement.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,” unless expressly specified otherwise.

In some embodiments, cements, cement components, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may be made of a material such as silicon nitride, which may alternatively be combined, in various embodiments, with other materials such as, for example, a polymer, a metal, an alloy, or the like. For instance, a disclosed device(s) may comprise a silicon nitride/PMMA cement, alone or in combination with a bone cement precursor and/or a Polyether Ether Ketone (PEEK), a titanium, a titanium alloy, or the like, or various combinations of the foregoing. The material may be formed by a process such as, for example, an active reductive process of a metal (e.g., titanium or titanium alloy) to increase the amount of nanoscaled texture to device surface(s), so as to increase promotion of bone growth and fusion.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it will be readily apparent that more than one component, device and/or article may be used in place of a single component, device and/or article. Similarly, where more than one component, device and/or article is described herein, it will be readily apparent that a single component, device and/or article may be used in place of the more than one component, device and/or article. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various components, devices, systems and methods for treating various anatomical structures of the spine and/or other areas of human and/or animal bodies. While the disclosed embodiments may be particularly well suited for use during surgical procedures for the repair, fixation and/or support of vertebrae, it should be understood that various other anatomical locations of the body may benefit from various features of the present invention, including for the repair of bones and for use in, for example, orthopedic surgery, including vertebrae repair, musculoskeletal reconstruction, fracture repair, hip and knee reconstruction, osseous augmentation procedures and oral/maxillofacial surgery.

Various embodiments herein encompass bone cement formulations that incorporate silicon nitride (i.e., Si₃N₄ and/or chemical analogues thereof) in their mixtures and/or composition, which may include the incorporation of silicon nitride powders, granules, particulates, portions, pebbles, blocks, layers and/or coatings of solids and/or particulates within the bone cement mixture. In various embodiments, the bone cement may be a liquid, paste, gel or dough, and preferably hardens to a substantially solid solidified material.

In at least one exemplary embodiment, a PMMA bone cement formulation comprising a powered MMA-styrene co-polymer and a reactive monomer such as methyl methacrylate can be combined with various percentages by weight and/or volume of a ceramic material such as a silicon nitride material, which when mixed and polymerized can result in a polymerized and/or “cured” block, implant and/or structure capable of implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

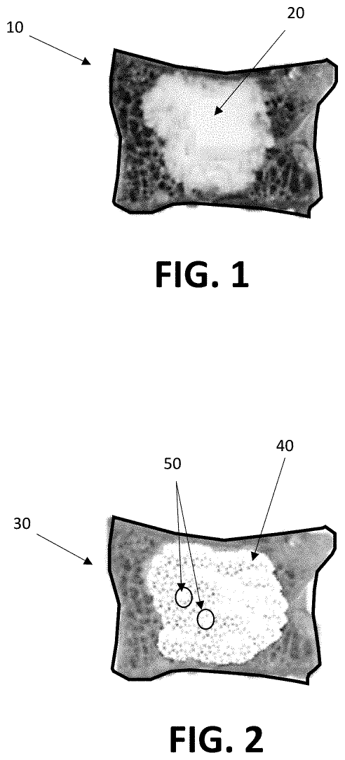

Historically, PMMA has been established as a very most material for fixation in joint replacement surgery. Polymerization of methyl methacrylate is a reaction that results in a doughy substance that self-cures in a short time. PMMA is made of a methyl methacrylate monomer precursor that polymerizes to form PMMA. There are a number of commercially manufactured PMMA cements available, each cement kit comprising an individually packaged granules and a liquid. The package typically comprises a powdered PMMA as its major constituent, together with a liquid vial which contains the monomer sub-unit, methyl methacrylate. Additionally, there are a number of other chemicals included to start and regulate the polymerization process (such as benzoyl peroxide). Additionally, opacifiers or oligomers of PMMA may also be contained. FIG. 1 depicts an exemplary vertebral bone 10 filled with a PMMA bone cement 20.

In at least one example, a PMMA bone cement formulation comprising a powered MMA-styrene co-polymer and a reactive monomer such as methyl methacrylate can be combined with various percentages by weight and/or volume of a powdered, granulated and/or fluidized silicon nitride material, which when mixed can create a flowable and/or moldable material which will desirably harden and/or polymerize into a variety of shapes, which can include injection of the flowable material through a syringe or tube into a void or opening to partially and/or fully fill the void or opening, wherein the material will subsequently harden and/or polymerize into a shape which can be defined by the cavity in which it sits. This could include the injection into various anatomical locations as well as injection and/or introduction into implants and/or other devices prior to, during and/or after their implantation into a targeted patient anatomy (i.e., such as within the graft chamber of an intervertebral fusion implant). As best seen in FIG. 2, this case include the injection of a PMMA/silicon nitride cement bolus 40 into a vertebra body 30, with the cement bolus 40 including a plurality of silicon nitride granules 50 therein.

In various embodiments, a settable bone cement of similar material could comprise a PMMA or other type of bone cement, in combination with silicon nitride and/or a resorbable granular material such as calcium phosphate or other material, which facilitates bone ingrowth, bone outgrowth and/or bone through-growth in varying amounts. Such a cement could provide the improved bacteriostatic properties of silicon nitride, and also allow for superior adhesions and/or anchoring of the cement to surrounding structures. Unlike typical bone cements, which may not interdigitate and/or which may be a source for bacterial infection, cements of the present invention inhibit and/or prevent the presence of bacteria within the bone cement bed. Moreover, unlike antibiotic-loaded bone cements, the bacteriostatic properties of silicon nitride are not anticipated to appreciably fade or diminish over time, and the present of silicon nitride within the cement mixture does not markedly weaken the strength and/or durability of the cured and/or polymerized cement.

In accordance with various aspects of the present subject matter, bone cements and/or other implants, devices and/or components thereof are described that incorporate silicon nitride (i.e., Si3N4 and/or chemical analogues thereof) in their construction, either in the entirety of the implant as well as components, portions, layers and/or surfaces thereof. In various embodiments, the silicon nitride material(s) will be highly osteo-inductive and/or osteoconductive and will desirably facilitate and/or promote fixation to adjacent living bone surfaces, while concurrently reducing and/or inhibiting periprosthetic infection and/or bacterial adhesion.

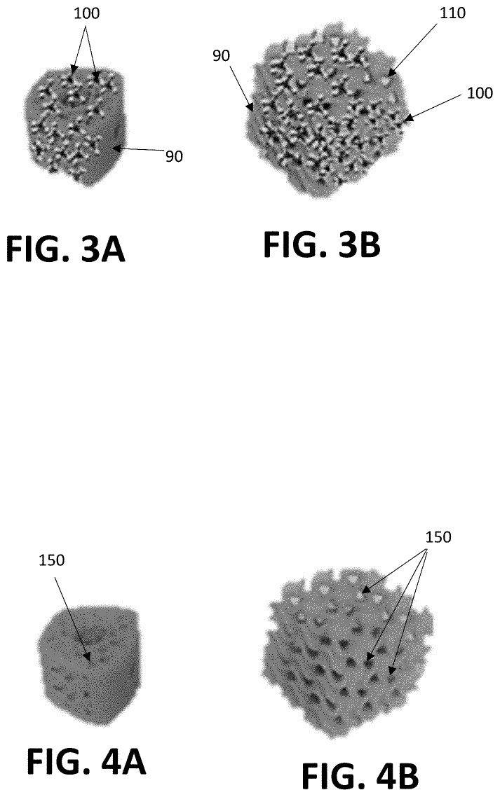

As best seen in FIGS. 3A and 3B, the settable silicon nitride cement will desirably comprise a PMMA base 90 with a plurality of silicon nitride granules 100 therein. In various embodiments, the granules can be mixed with and suspended within the curing and/or cured PMMA, with various portions of the silicon nitride granules exposed to the surrounding anatomy. In various embodiments, such as shown in FIG. 3B, the PMMA base 90 can further optionally include openings and/or voids 110 formed therein. FIGS. 4A and 4B depict the settable cement blocks of FIGS. 3A and 3B after partial resorption of silicon nitride granules near the surface of the blocks, wherein additional macro pores 150 have been formed as some of the silicon nitride granules have been resorbed and/or remodeled by the patient.

In various applications, the utility of silicon nitride as a component of medical implants may be further enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

The various cements, mixtures, devices, implants and/or components thereof disclosed herein can incorporate a silicon nitride material (i.e., Si3N4 and/or chemical analogues thereof) in their construction, either in one or more of a two part mixture, as well as within the entirety of an implant as well as components, portions, layers and/or surfaces thereof. The incorporation of silicon nitride as a component material for spinal implants can provide significant improvements over existing implant materials and material designs currently available, as the silicon nitride material(s) will be highly osteo-inductive and/or osteoconductive and will desirably facilitate and/or promote implant fixation to adjacent living bone surfaces, while concurrently reducing and/or inhibiting periprosthetic infection and/or bacterial adhesion to the surfaces and/or interior portions thereof.

Silicon nitride (Si3N4) and its various analogs can impart both antibacterial and osteogenic properties to an implant, including to mixtures containing Si2N4 and/or bulk Si3N4 as well as to implants coated with layers of Si3N4 of varying thicknesses. In bone replacement as well as prosthetic joint fusion and/or replacement, osseous fixation of implants through direct bone ingrowth (i.e., cementless fixation) is often preferred, and such is often attempted using various surface treatments and/or the incorporation of porous surface layers (i.e., porous Ti6Al4V alloy) on one or more bone-facing surfaces of an implant. Silicon nitride surfaces and/or interior portions express reactive nitrogen species (RNS) that promote cell differentiation and osteogenesis, while resisting both gram-positive and gram-negative bacteria. This dual advantage of RNS in terms of promoting osteogenesis, while discouraging bacterial proliferation, can be of significant utility in a variety of implant designs.

Desirably, the inclusion of silicon nitride components into a given cement mixture will encompass the use of granularized and/or powdered silicon nitride, as well as bulk silicon nitride, as well as implants incorporating other materials that may also include silicon nitride components and/or layers therein, with the silicon nitride becoming an active agent of bone fusion. RNS such as N2O, NO, and —OONO are highly effective biocidal agents, and the unique surface chemistries of Si3N4 facilitate its activity as an exogenous NO donor. Spontaneous RNS elution from Si3N4 discourages surface bacterial adhesion and activity, and unlike other direct eluting sources of exogenous NO, Si3N4 elutes mainly NH4+ and a small fraction of NH3 ions at physiological pH, because of surface hydrolysis and homolytic cleavage of the Si—N covalent bond. Ammonium NH4+ can enter the cytoplasmic space of cells in controlled concentrations and through specific transporters, and is a nutrient used by cells to synthesize building-block proteins for enzymes and genetic compounds, thus sustaining cell differentiation and proliferation. Together with the leaching of orthosilicic acid and related compounds, NH4+ promotes osteoblast synthesis of bone tissue and stimulates collagen type 1 synthesis in human osteoblasts. Conversely, highly volatile ammonia NH3 can freely penetrate the external membrane and directly target the stability of DNA/RNA structures in bacterial cells. However, the release of unpaired electrons from the mitochondria in eukaryotic cells activates a cascade of consecutive reactions, which starts with NH3 oxidation into hydroxylamine NH2OH (ammonia monooxygenase) along with an additional reductant contribution leading to further oxidation into NO2− nitrite through a process of hydroxylamine oxidoreductase. This latter process involves nitric oxide NO formation. In Si3N4, the elution kinetics of such nitrogen species is slow but continuous, thus providing long-term efficacy against bacterial colonies including mutants (which, unlike eukaryotic cells, lack mitochondria). However, when slowly delivered, NO radicals have been shown to act in an efficient signaling pathway leading to enhanced differentiation and osteogenic activity of human osteoblasts. Desirably, Si3N4 materials can confer resistance against adhesion of both Gram-positive and Gram-negative bacteria, while stimulating osteoblasts to deposit more bone tissue, and of higher quality.

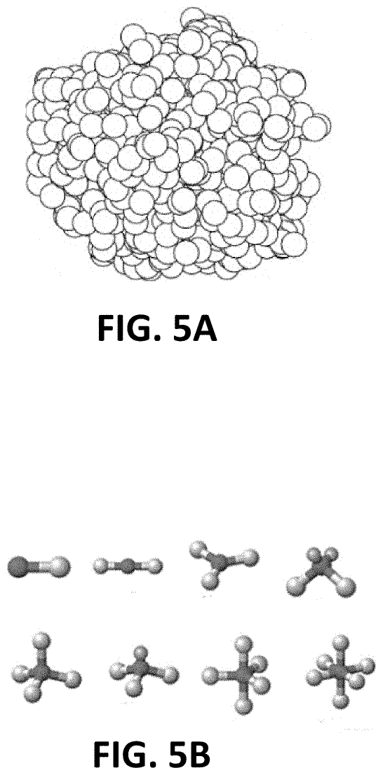

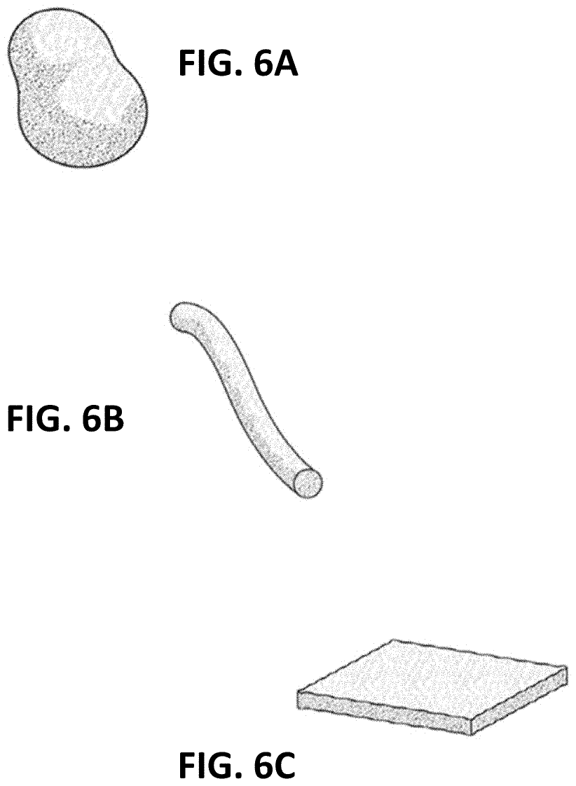

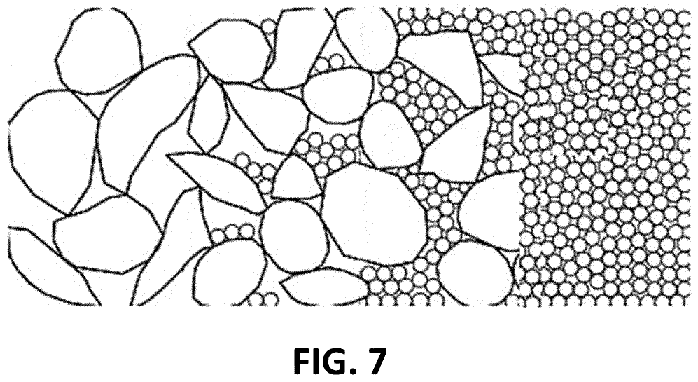

In another exemplary embodiment, disclosed is a bone cement which has both improved structural properties and improved osteoconductivity to regenerate and heal the host bone tissue. In this embodiment, the distribution of a granulated microporous ceramic material such as silicon nitride within bone cement will desirably provide improved structural properties for the hardened bone cement, whilst the microporous structure of the ceramic material granules allows host tissue to bind and regenerate around and within the bone cement-ceramic material mixture. In some embodiments the ceramic material granules may comprise a single average size granule or granule distribution (See FIG. 5A), while in other embodiment, the granule size may be widely distributed and/or essentially random within a range of sizes (See FIG. 7). In another alternative embodiment, at least two different preselected sizes, or ranges of sizes, of granulated material can be used, e.g. in a similar manner to sand and gravel being used with cement to make concrete. The different size of the silicon nitride “sand and gravel” may be helpful in improving the strength of the material. Preferably, the ceramic material will be generally evenly distributed throughout a cross-section of the hardened bone cement, that is substantially without clumps of ceramic material forming. If desired, the various silicon nitride granules may comprise a variety of different shapes, including rounded particles, irregularly shaped particles (see FIG. 6A), elongated particles, fibers or “strings” (see FIG. 6B), flattened or planar particles (see FIG. 6C), or other shapes, or any combination thereof. In many cases, multiple shapes and/or sizes of particles may provide for optimized packing and/or density of the silicon nitride material for certain applications.

If desired, a variety of sizes and/or shapes of silicon nitride granules and/or particles may be utilized in various embodiments of the present invention, which can include particles and/or microparticles that form a variety of geometric bonds and/or matrix shapes, including linear, trigonal planar, bent or angular, tetrahedral, trigonal pyramidal, trigonal bipyramidal, octahedral, and/or other shapes, including those depicted in FIG. 5B.

In various embodiments, the individual granules of the silicon nitride material may have micropores. Preferably, the micropores are interconnecting. They are preferably not confined to the surface of the granules but are found substantially throughout the cross-section of the granules. Preferably, the diameter of the granule particles is between 10 μm and 1 mm, preferably 400 μm and 1000 μm, especially 500-900 μm, 500-800 μm or 600-700 μm. The ceramic material granules may be formed from a fused block of biomaterial by milling or grinding using, for example, a ball mill, and the size of the granules may be adjusted using, e.g. one or more sieves. In this manner, two or more different sized particles, or ranges of sizes of particles, may be obtained from a single “run” of the ball mill, if desired.

Where the silicon nitride granule size within a given cement formulation is distributed between two different preselected sizes, or ranges of sizes, some embodiments may comprise a mixture of small and large granules. For example, the small granules may have a size range of 10 μm to 500 μm, especially 50 to 350 μm, most preferably 100 to 250 μm diameter, while the large granules may have a diameter of 250 μm to 1.5 mm, especially 500 μm to 1 mm, most preferably 600 μm to 800 μm.

In various embodiments, the bone cement may comprise a mixture of a PMMA bone cement or a bone cement precursor with the ceramic material granules. That is, in the solidified bone substitute, the bone cement forms a matrix that binds together the ceramic material granules, in a similar manner to the lime or cement constituting the cementing material that binds together the sand and aggregate in a mortar or concrete. The term “bone cement precursor” is intended to mean one or more compounds which, upon curing or solidifying, form a substantially solid bone cement matrix. For example, with PMMA, methyl methacrylate monomer is polymerized to form the PMMA bone cement. The monomer is a bone cement precursor. Similarly, to form inorganic materials, such as calcium phosphate, bone cement may be formed, for example, by mixing dicalcium phosphate dihydrate with tetracalcium phosphate. These two compounds can act as precursors to the final bone cement. Upon wetting, the two materials react and solidify to form a solidified bone cement. In at least one exemplary embodiment, the bone cement, which may be made from the bone cement precursor, is a polymeric material or an inorganic ceramic material. Preferably, the organic material is a poly (meth) acrylate material, such as PMMA or PAA (polyacrylic acid).

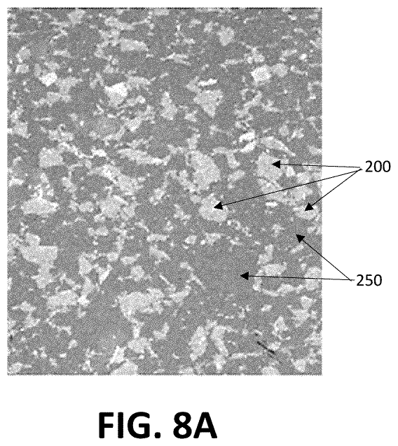

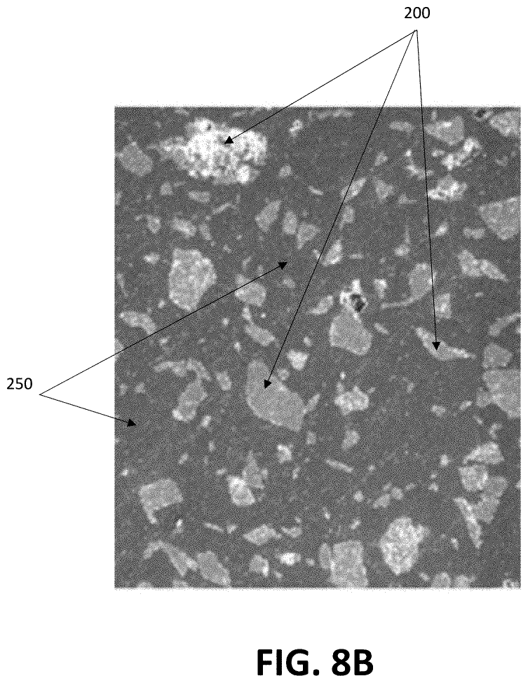

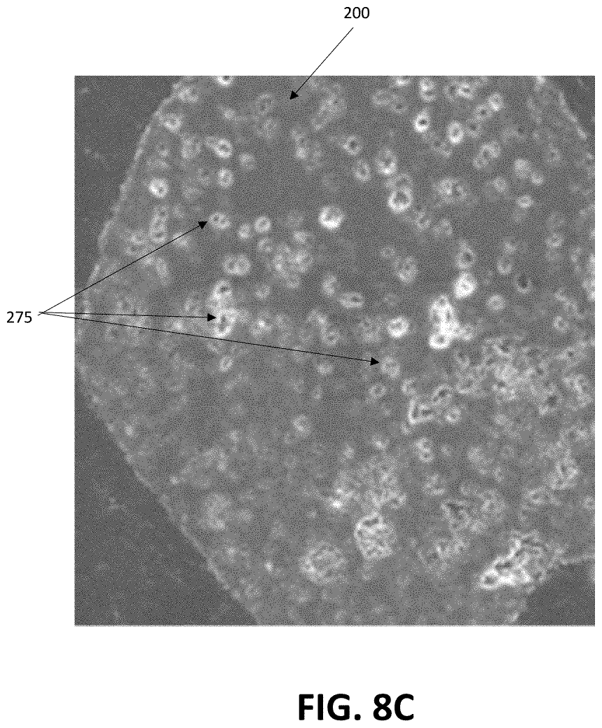

FIGS. 8A and 8B depicts scanning electron microscope (SEM) pictures of an exemplary PMMA/ceramic cement. In this embodiment the ceramic can comprise granules of a silicon nitride material 200, which is adhered and/or held within a cured PMMA matrix 250. Where the bone cement material is desirably injectable through a smaller diameter opening and/or incision, the use of bulk and/or large granules of silicon nitride (or other material) implants may not be desirous and/or may be impractical. In such embodiments, it may be desirable to incorporate silicon nitride in a formable and/or curable form, which may include the incorporation of silicon nitride with other materials such as curable bone cements. In such a case it may be desirable for the silicon nitride to be provided in small granular and/or powdered form, to allow easy mixing with the bone cement constituents. Desirably, the size of the granules allows it to be injected through the bore of a needle from, for example, a syringe, into position on the surface of a bone under repair. For example, the preferable maximum size of silicon nitride granules and/or particles in such an application may be 0.1 mm, or 0.5 mm, or 1.0 mm, or 1.5 mm.

As best seen in FIG. 8C, the silicon nitride granules 200 can each include a plurality of micropores 275 formed therein, with the micropores of varying shapes and/or sized within an individual granule. In various aspects of the invention, the ceramic granules will each include a plurality of micropores formed therein. In some aspects, the invention provides: a bone substitute comprising a mixture of a bone cement or a bone cement precursor and silicon nitride material granules, the silicon nitride granules having a plurality of micropores of an average diameter of between 1 μm and 10 μm and/or between 10 μm and 50 μm and/or between 50 μm and 100 μm. Of course, smaller and/or larger pore sizes within the granules may have particular utility in certain applications.

In various embodiments, the PMMA described herein may be replaced by bisphenol-alpha-glycidyl methacrylate resin (BIS-GNA) to form alternative organic bone cements. Alternatively, PAA may be used in combination with aluminosilicate glass to form silicate cement or “glass ionomer cement” (GIC).

If desired, the bone cement mixtures described herein may additionally comprise one or more additional materials such as accelerators or regulators in order to control the curing of the bone cement. Catalysts of organic polymerization reactions include peroxides, such as benzyl peroxide. Accelerators of inorganic cements are known, for example disodium hydrogen phosphate is known to be used as an accelerator. Opacifiers or colorants may also be included. Additionally, polystyrene may be included as necessary to improve the handling of the properties of the cement.

In various embodiments, the volume and/or weight ratio of silicon nitride to cement may be 1000:1, 100:1, 50:1, 10:1, 5:1, 2:1, 1:1, 1:2, 1:5, 1:10, 1:50. 1:100 and/or 1:1000 or lesser/greater, and/or any ranges between any combination of the above.

If desired, the bone cement mixture may additionally comprise one or more pharmaceutically and/or biologically active compounds. These may be incorporated into the micropores and/or mid-pores and in use may be used to stimulate cell growth around and into the biomaterial. For example, growth factors, such as transforming growth factor (TGF-βI), bone morphogenetic protein (BMP-2) or osteogenic protein (OP-I) maybe incorporated into the biomaterial. Further materials such as enzymes, vitamins (including Vitamin D) and trace material such as zinc (for example in the form of salt) may also be incorporated.

The ceramic material used to produce the granules may be any non-toxic ceramic known in the art, but in various embodiments will comprise a silicon nitride material and/or its chemical analogues.

In another exemplary embodiment, the PMMA may desirably be located primarily between the granules of silicon nitride, such that the PMMA adheres the adjacent granules together without completely encompassing each of the granules. In such a case, the resulting silicon nitride “block” or implant may have a spongy or swiss-cheese-like appearance. In this embodiment, a liquid monomer may be distributed within some of the pores of the silicon nitride granules, with powdered PMMA later mixed with and/or “dusted over” the granules to cause polymerization of the PMMA between the silicon nitride granules (i.e., similar to a “gluing” agent). Coating of the silicon nitride particles in this manner may improve the distribution of the particles through the finely fused product and produce a substantially uniform product with substantially evenly distributed micropores. Where the coating agent is liquid, for example PEG, simply mixing the ceramic particles in the coating agent may coat the particles. Alternatively, some coating agents, such as the starch and agar coating agents may be mixed with an inert liquid, such as water, in a granules form, and heated to allow the starch or agar to form a polymer coating around the particles. Heating liquids containing starch can cause the starch to polymerize and thicken the liquid in a similar manner to adding corn flour to thicken gravy when cooking.

In various embodiments, disclosed are biomaterials obtainable by various manufacturing processes. Bone implants, dental implants or ear, nose or throat (ENT) implants comprising both substitute materials according to the invention are also provided. Additionally, the invention provides the use of the bone substitute as a bone replacement, in dental implants or maxillofacial repair materials for the repair of bone breaks or fractures, osteoporotic bone, intervertebral space implants, and/or as a bone glue or putty or a load bearing surface on a bone. Furthermore, the bone substitute may be used, for example, to create attachment points for devices such as screws or plates.

In various embodiments, a bone cement material can be made by mixing a bone cement precursor, as defined above, together with the silicon nitride material, as defined above, to form a paste and the bone precursor is caused to convert to a substantially solid bone substitute material comprising a bone cement and a ceramic material by, for example, the use of a catalyst or the reaction of the bone precursor materials. While PMMA may be a preferred material in many embodiments, the disclosed inventions similarly contemplate the use of other bone cement materials instead of PMMA. The bone cement precursors may be obtained from manufacturers and used according to the manufacturers' instructions, with the addition of the silicon nitride material granules which is mixed and dispersed within the bone cement prior to its setting.

In one exemplary embodiment, a silicon nitride material can be pulverized, for example using a ball mill or other milling machinery. The size of the resulting silicon nitride granules may be adjusted, for example, by sieving through a mesh of the desired size to regulate the size of the granules. The granules can then be mixed with a bone cement precursor, in a similar manner to adding aggregate to cement to form concrete. The cement precursor may be PMMA, which can be purchased from a variety of well-known manufacturers. Cement kits usually consist of an individually packed granules and a liquid which is typically sterilized by gamma irradiation and ultra-filtration. The packaged granules can contain PMMA as its major constituent. The liquid contains the monomer sub-unit, methyl-methacrylate. Additionally, one or more other ingredients may include chemicals that are responsible for the polymerization reaction rate, as well as the handling properties of the cement and the resistance to degradation. An initiator polymerization, such as benzoyl peroxide, may be provided to start the polymerization reaction. Additionally, polystyrene may be included as this improved the handling of the properties of the cement.

In the exemplary embodiment, a weight ratio of precursor to the silicon nitride material granules may be between 100:1 to 1:100, especially 10:1 to 1:10, and more preferably 1:1.

In various embodiments the silicon nitride granules and/or cement components thereof can be formed using a variety of techniques, including by compressing, milling and firing silicon nitride powder, as well as by extruding silicon nitride into sheet, tube, pipe and/or thread form (which may be further processed into thread or “rope” by braiding and/or other techniques). Silicon nitride shapes may also be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, silicon nitride may even be formed using curing or other light/energy activation techniques, such as where a slurry of liquid polymer and silicon nitride particles may be UV cured to create a 3-dimensional structure and/or layer containing silicon nitride. In various embodiments, silicone nitride may be utilized in block form, in sheets, columns and bars, in cable or braided form, in mesh form, in a textured surface coating, in powder form, in granular form, in gel, in putty, in foams and/or as a surface filler and/or coating. In some cases, a surface layer of silicon nitride cement may be formed, placed and/or deposited on an external and/or internal surface of an implant.

Once implanted in a desired location, the silicon nitride cement will desirably be highly osteo-inductive and/or osteoconductive and will desirably facilitate and/or promote fixation of the cement and/or any implants used therewith to adjacent living bone surfaces, while concurrently reducing and/or inhibiting periprosthetic infection and/or bacterial adhesion to the surfaces and/or interior portions of the cement.

If desired, a bone implant could be constructed from a variety of modular components, including at least one “modular” component comprising a silicon nitride cement. If desired, such an implant and/or the components thereof could be provided in a kit form for selection and/or assembly in a surgical theatre and/or in situ during a surgical procedure. If desired, various components may be removable and replaceable. If desired, the silicon nitride cement could be provided in a fully cured form, such as in part of the implant, or could be provided in a mixable and/or flowable form, wherein the cement can cure during the surgical procedures.

Various surgical methods for preparing anatomical surfaces and/or for implanting or placing silicon nitride cements are also described, including the insertion and placement of such cement between adjacent vertebrae of the spine as well as within bones and/or between other joint surfaces.

In accordance with another aspect of the present subject matter, various methods for manufacturing a silicon nitride cement and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

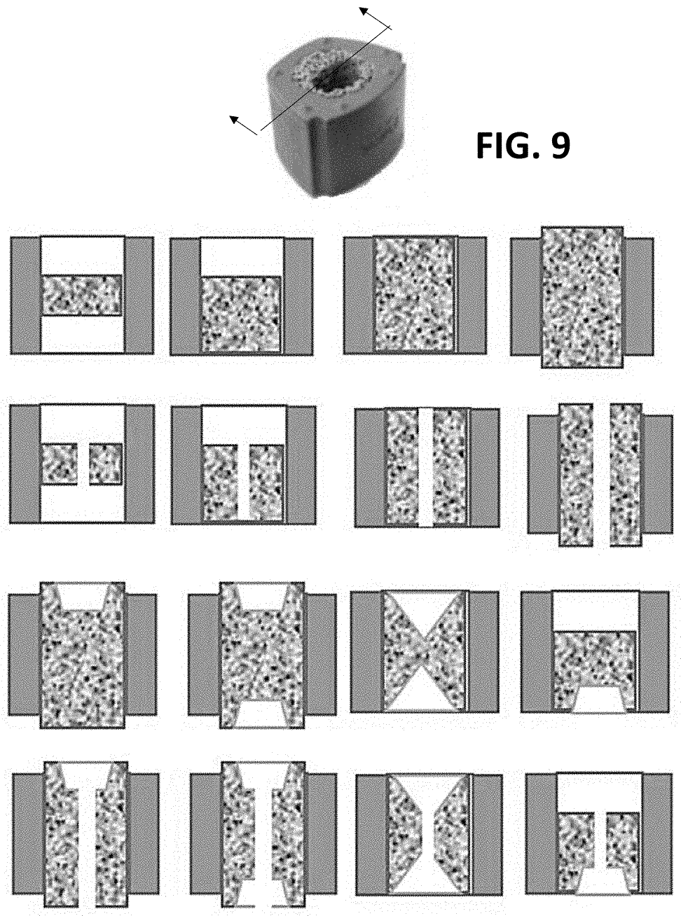

As previously noted, the various bone cement formulations, bone implants and/or components thereof disclosed herein can incorporate a silicon nitride material (i.e., Si₃N₄ and/or chemical analogues thereof) in their construction, either in the entirety of the implant as well as components, portions, layers, fillings and/or surfaces thereof. The incorporation of silicon nitride as a component material for spinal or other implants can provide significant improvements over existing implant materials and material designs currently available, as the silicon nitride material(s) will desirably be highly osteo-inductive and/or osteoconductive and will facilitate and/or promote implant fixation to adjacent living bone surfaces, while concurrently reducing and/or inhibiting periprosthetic infection and/or bacterial adhesion to the surfaces and/or interior portions of the implant. In various embodiments, materials including silicon nitride materials of differing compositions and/or states (i.e., solid, liquid and/or flowable or moldable “slurry” states, for example) could be utilized in a single implant and/or portions thereof, including the use of solid silicon nitride for an arthroplasty cage implant, with a moldable silicon nitride “paste” placed within a centrally positioned “graft chamber” of the implant. If desired, an implant could include some portion or insert formed from a silicon nitride cement, wherein the silicon nitride or similar component could extend completely through an implant, or only extend partially into and/or out of an implant. For example, FIG. 9 depicts various cross-sectional views of spinal implants with various exemplary silicon nitride insert geometries formed therein, which can include the introduction of such silicon nitride materials in an uncured form which can then cure in situ, if desired.

In various embodiments the disclosed implants may incorporate materials such as silicon nitride that are “phase stable” to a desired degree. For example, various embodiments may desirably withstand standard autoclave sterilization conditions such as 120° C. 1 atmosphere steam for up to 100 hours of time, with no appreciable change in phase composition, no appreciable change in flexural strength and an inherently stable microstructure. Moreover, such materials could desirably provide favorable imaging characteristics, such as high levels of radiolucency and/or no significant MRI or CT scan artifacts.

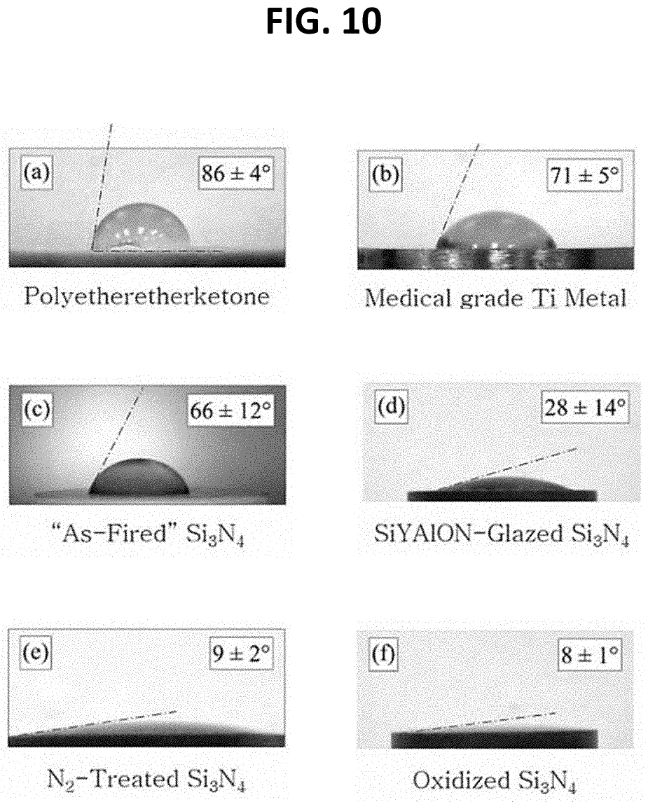

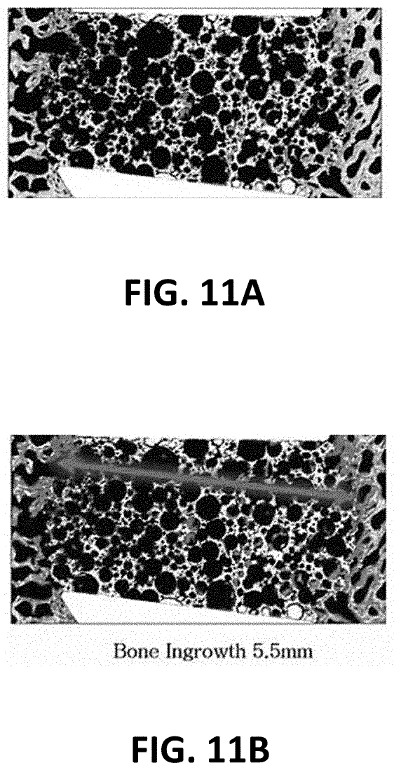

FIG. 10 depicts exemplary degrees of hydrophobicity for various medical grade materials, including silicon nitride in various forms. As shown, silicon nitride is much less resistant to water penetration than other materials, which can be a highly desirably characteristic in many applications. In many applications, a porous cement formed from silicon nitride can induce neovascularization within the porous sections of the implant, including internal pores colonized with mineralized bone to a depth exceeding 5.5 mm, such as depicted in FIGS. 11A and 11B.

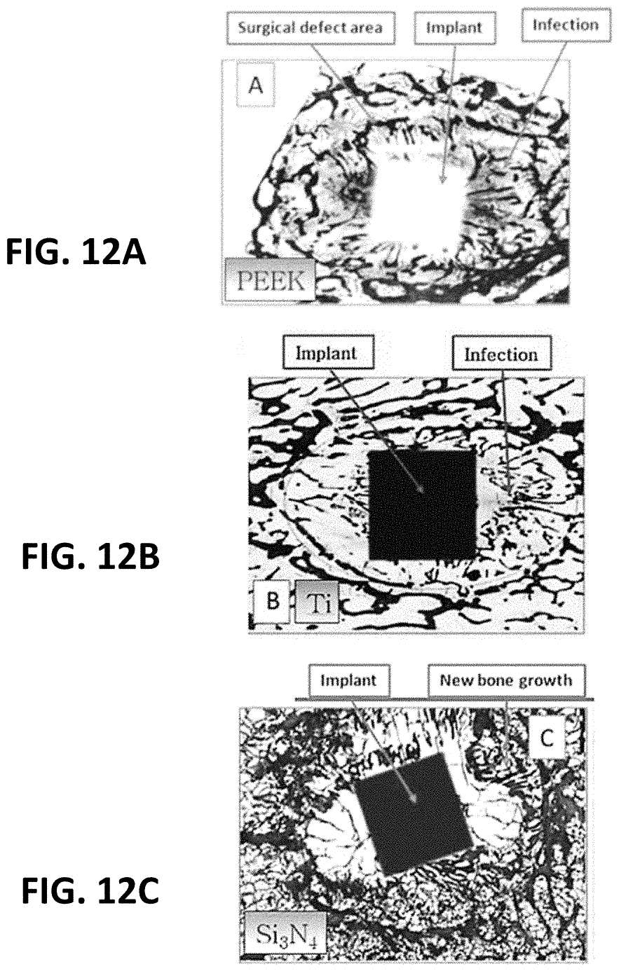

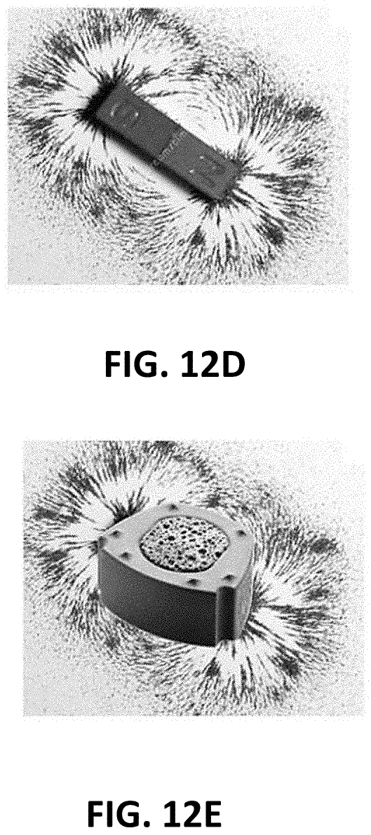

FIGS. 12A through 12C depict three exemplary implants, one each made of PEEK, Titanium and a third incorporating a silicon nitride material (such as a silicon nitride cement material as described herein) and their effects on adjacent living bone. As shown in FIG. 12A, a PEEK implant may often be accompanied by surgical bone defects that do not fill in with new bone over time, as well as potential infection sites proximate to the implant that may be difficult or impossible to resolve (potentially necessitating implant removal in some cases). In a similar manner, as shown in FIG. 12B, bone infection sites near titanium implants can also be difficult or impossible to resolve, and may similarly necessitate implant removal. However, with an implant incorporating a silicon nitride material, such as shown in FIG. 12C, the surface chemistry of the implant actively destroys infectious bacterial agents, and also induces new bone growth immediately upon implantation. In essence, the effect of the silicon nitride material on new bone growth acts like a magnet on ferrous materials (see FIG. 12D), actively “drawing” new bone near and into the implant (see FIG. 12E).

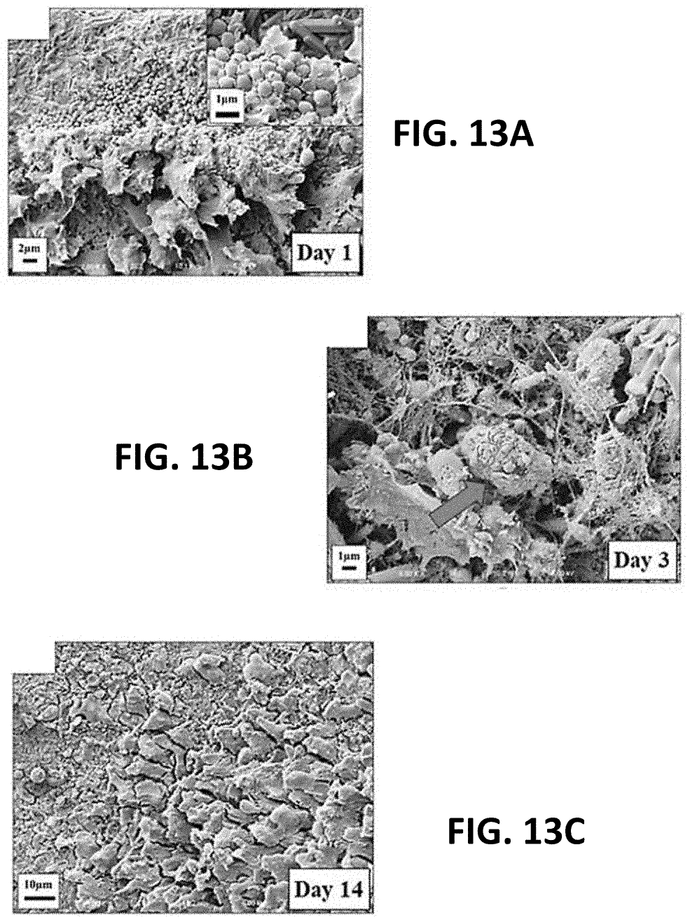

Another significant advantage of using silicon nitride materials in bone implants is the anti-bacterial effects of the material on infectious agents. As best seen in FIG. 13A, upon implantation a silicon nitride surface can induce an inflammatory response action which attacks bacterial biofilms near the implant. This reaction can also induce the elevation of bacterial pods above the implant surface by fibrin cables (see FIG. 13B). Eventually the bacteria in the vicinity of the silicon nitride implant surfaces will be cleared by macrophage action, along with the formation of osteoblastic-like cells (See FIG. 13C). In various experiments involving comparisons between standard implants and silicon nitride implants (both bulk and silicon nitride coated implants of standard materials), cell viability data in (which were determined at exposure times of 24 and 48 hours, showed the existence of a larger population of bacteria on the standard medical materials as compared to Si₃N₄ implants (both coated and bulk). A statistically validated decreasing trend for the bacterial population with time was detected on both coated and bulk substrates, with a highest decrease rate on Si₃N4-coated substrates. Moreover, the fraction of dead bacteria at 48 h was negligible on the standard implants, while almost the totality of bacteria underwent lysis on the Si₃N₄ substrates. In addition, optical density data provided a direct assessment of the high efficacy of the Si₃N₄ surfaces in reducing bacterial adhesion.

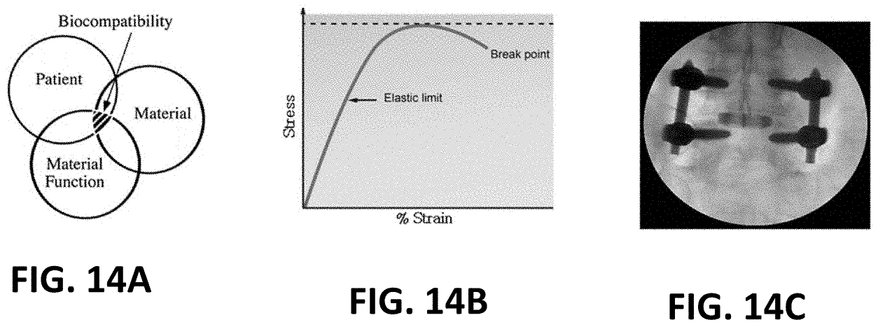

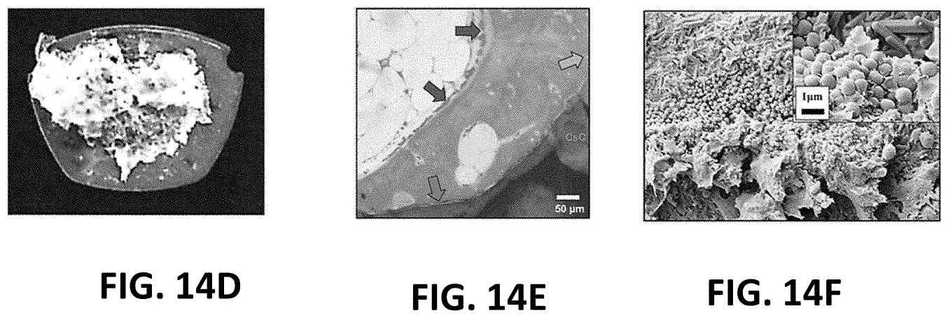

In various embodiments, the disclosed silicon nitride cements can provide various combinations of significant advantages and desirable attributes of an abiotic spinal spacer or similar implant, such as one or more of the following: biocompatibility (FIG. 14A), mechanical integrity (FIG. 14B), radiological traceability (FIG. 14C), osteoconductivity (FIG. 14D), osteoinductivity (FIG. 14E) and/or bacteriostasis (FIG. 14F). In various embodiments, silicon nitride materials including the silicon nitride cements disclosed herein can be incorporated into a variety of implants and implant-like materials, including (1) orthopedic bone fusion implants (i.e., screws, cages, cables, rods, plugs, pins), (2) dental implants, (3) cranial/maxillofacial implants, (4) extremity implants, (5) hip and joint implants, (6) bone cements, powders, putties, gels, foams, meshes, cables, braided elements, and (7) bone anchoring elements and/or features.

In various embodiments, a silicon nitride cement can be manufactured and/or molded into various shapes and/or sizes, which could include placement and/or incorporation into and/or around a shaft or other feature of a bone screw or other surgical implant. In some cases, because the silicon nitride material may shrink or otherwise deform during portions of the manufacturing and/or curing process, it may be desirable that the implant design features accommodate potential changes in the dimensions and/or density of the cement. In at least one exemplary embodiment, a silicon nitride cement may be mixed and molded in a sleeve or other shape, with a corresponding metal implant shape receiving the silicon nitride cement (either in a fully, partially and/or uncured condition).

In various embodiments, a surgical tool kit could include a cement comprising silicon nitride as one or more modular components for the system, including fully formed individual silicon nitride components and/or uncured cements, if desired. The various components of these systems could optionally be provided in kit form, with a medical practitioner having the option to select an appropriately sized and/or shaped implant and/or modular components to address a desired surgical situation.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

Of course, method(s) for manufacturing the various cement formulations, silicon nitride components and/or surgical devices and related components and implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A hybrid bone cement comprising a mixture of a bone cement or a bone cement precursor and a plurality of silicon nitride granules, the plurality of silicon nitride granules having an average diameter of 10 μm to 1.5 mm.

2. The hybrid bone cement of claim 1, wherein the plurality of silicon nitride granules comprises at least two different preselected sizes, or range of sizes, of silicon nitride granules.

3. The hybrid bone cement of claim 2, wherein the two different preselected sizes of silicon nitride granules comprise a larger granule size of 500 μm to 1.5 mm and a smaller granule size of 10 μm to 500 μm.

4. The hybrid bone cement of claim 1, wherein the silicon nitride granules comprise a plurality of substantially spherical granules.

5. The hybrid bone cement of claim 1, wherein the bone cement, or the bone cement precursor on conversion to bone cement comprises PMMA, PAA, a calcium phosphate, or calcium sulphate.

6. The hybrid bone cement of claim 1, further comprising one or more biologically or pharmaceutically active compounds.

7. The hybrid bone cement of claim 6, wherein the pharmaceutically active compound is a cell growth factor or bone morphogenic protein.

8. The hybrid bone cement of claim 1, wherein the bone cement comprises a two-part mixture of powdered PMMA and a liquid monomer.

9. The hybrid bone cement of claim 8, wherein the silicon nitride granules are mixed with a dispersing agent prior to the powdered PMMA and liquid monomer being mixed together.

10. The hybrid bone cement of claim 8, wherein a weight ratio of the bone cement to the silicon nitride granules is approximately 1:1.

11. The hybrid bone cement of claim 8, wherein a weight ratio of the bone cement to the silicon nitride granules is at least 10:1 or greater.

12. The hybrid bone cement of claim 8, wherein a weight ratio of the bone cement to the silicon nitride granules is at least 1:10 or greater.

13. The hybrid bone cement of claim 8, wherein a volume ratio of the bone cement to the silicon nitride granules is approximately 1:1.

14. The hybrid bone cement of claim 8, wherein a volume ratio of the bone cement to the silicon nitride granules is at least 10:1 or greater.

15. The hybrid bone cement of claim 8, wherein a volume ratio of the bone cement to the silicon nitride granules is at least 1:10 or greater.
